(19) United States
(12) Patent Application Publication
(10) Pub. No.: US 2017/0281076 A1
(43) Pub. Date: Oct. 5, 2017

(54) SLEEP STATE MONITORING SYSTEM BASED ON PULSE WAVE MEASUREMENT

(71) Applicant: ACT MEDICAL SERVICE CO., LTD., Fukushima (JP)

(72) Inventors: Shinichi TAKAHASHI, Fukushima (JP); Shintaro CHIBA, Tokyo (JP); Asako YAGI, Kanagawa (JP)

(21) Appl. No.: 15/508,304

(22) PCT Filed: Jul. 16, 2015
(86) PCT No.: PCT/JP2015/070448

§ 371 Date: Mar. 2, 2017

(30) Foreign Application Priority Data
Sep. 3, 2014 (JP) 2014-179537

(51) Int. Cl. A61B 5/00; A61B 5/021

(52) U.S. Cl. CPC A61B 5/4809; A61B 5/02108; A61B 5/6826

(57) ABSTRACT

A sleep state monitoring system is provided a pressure sensor, an optical sensor, and a controller. The pressure sensor detects a change in pressure of a pulse wave propagating through a blood vessel to measure a voltage value as a first voltage value of arterial pressure, and the optical sensor detects a change in pressure of a pulse wave propagating through the blood vessel by use of an optical signal to measure a voltage value as a second voltage value of a vascular pulse wave signal. The controller determines a sleep state of the human body, based on the first voltage value and the second voltage value.

### TECHNICAL FIELD

The present invention relates to a sleep state monitoring system for determining a sleep state by use of, for example, a radial arterial pressure measuring system or a radial arterial wave measuring system, and a fingertip vascular pulse wave measuring system. In particular, the present invention relates to a sleep state monitoring system for determining a sleep state by use of a radial arterial pressure measuring system for measuring radial arterial pressure or a radial arterial wave measuring system for measuring radial arterial wave, and a fingertip vascular pulse wave measuring system for acquiring a pulsation waveform of a fingertip blood vessel (hereinafter, referred to as a fingertip pulse wave) by use of an optical signal to perform fingertip pulse wave measurement.

### BACKGROUND ART

PSG (polysomnography, including brain wave measurement), which is an essential test for diagnosis of sleep apnea syndrome (SAS), necessitates hospitalization and a high-priced measurement instrument (see, for example, Patent Documents 1 and 2), and is required to have high analytical capability, and hence there are not many facilities capable of conducting PSG.

### PRIOR ART DOCUMENTS

### Patent Documents

[Patent Document 1] Japanese Patent Laid-open Publication No. JP2013-081691A
[Patent Document 2] Japanese Patent Laid-open Publication No. JP2014-008159A

### SUMMARY OF THE INVENTION

### Problems to be Solved by the Invention

In addition, simple sleep monitoring for monitoring only a respiratory state, which is operable at home and has also been performed in general medical institutions, has the following drawbacks.

(1) Since brain waves are not measured, it is not possible to decide whether breathing during sleep is monitored.

(2) Since sensors at a mouth and a nose of a face are easy to come off, the accuracy of data at home is low.

(3) Acquired data is poor in content.

In all-night PSG, since a sleep stage, such as sleep onset, waking after sleep onset, or waking, can be determined by measuring brain waves, it is possible to measure total sleep time obtained by removing wakeful time from recording time. However in the simple sleep test apparatus, brain waves are not measured and thus the total sleep time cannot be measured, which has been problematic.

An object of the present invention is to solve the above problems and to provide a sleep state monitoring system having a simple apparatus configuration as compared with the prior art and capable of determining a sleep state with higher accuracy.

### Means for Dissolving the Problems

According to the present invention, there is provided a sleep state monitoring system including first and second sensors and control means. The first sensor is one of a pressure sensor and an optical sensor. The first sensor of the pressure sensor is provided via a skin on a blood vessel of an aortic portion of a human body, and detects a change in pressure of a pulse wave propagating through the blood vessel to measure a voltage value as a first voltage value of arterial pressure. The first sensor of the optical sensor is provided via the skin on the blood vessel of the aortic portion of the human body and detecting a pulse wave propagating through the blood vessel to measure a voltage value as a first voltage value of blood vessel pressure. The second sensor is an optical sensor, and the second sensor is provided via a skin on a peripheral blood vessel of the human body and detects a change in pressure of a pulse wave propagating through the blood vessel by use of an optical signal to measure a voltage value as a second voltage value of a vascular pulse wave signal. The control means is configured to determine a sleep state of the human body, based on the first voltage value and the second voltage value. The control means

(A) determines that the human body is in non-wakefulness when the first voltage value and the second voltage value increase and decrease by amounts of increase and decrease within respective predetermined threshold ranges for a predetermined time interval, and

(B) determines that the human body is in wakefulness when the first voltage value increases by an amount of increase equal or larger than a predetermined first threshold and the second voltage value decreases by an amount of decrease equal to or smaller than a predetermined second threshold for the time interval.

In the sleep state monitoring system, the control means

(C) determines that the human body is in a state of minute variation of brain waves when the first voltage value increases and decreases by amounts of increase and decrease within the predetermined threshold ranges and the second voltage value decreases by an amount of decrease equal to or smaller than the predetermined second threshold range for the time interval.

In addition, in the sleep state monitoring system, the blood vessel of the aortic portion of the human body is a blood vessel of a radial portion of the human body, and the peripheral blood vessel of the human body is a blood vessel of a fingertip portion of the human body.

Further, the sleep state monitoring system further includes notification means that notifies the determination result.

Still further, in the sleep state monitoring system, each of the pressure sensors is a MEMS (Micro Electro Mechanical Systems) pressure sensor that detects, as a change in resistance value, a change in pressure of a pulse wave propagating through the blood vessel.

Further, in the sleep state monitoring system, the pressure sensor has a first space on a pressure detection surface side of a diaphragm, and a diaphragm that detects pressure by use of a pressure detection surface facing the first space, and outputs an electric signal corresponding to the detected pressure The sleep state monitoring system includes first and second films. The first film sheet supports the pressure sensor and placed in contact with a portion to be measured, and the first film sheet has a second space being communicated with the first space and larger than the first space, and having a size in a parallel direction to the pressure detection surface. The second film sheet has a third space having a size in the parallel direction to the pressure detection surface, and the second film sheet is provided for positioning the pressure sensor in the portion to be measured. The second film sheet is placed such that a region of the measured portion is located in the third space before the pressure sensor is placed in the portion to be measured.

Still further, in the sleep state monitoring system, the optical sensor is an optical sensor configured using an optical probe circuit, and the optical probe circuit includes an optical probe, a drive circuit, and a detection circuit. The optical probe includes a light-emitting element that emits light to a blood vessel via a skin, and a light-receiving element that receives, via a skin, reflected light from the blood vessel and transmitted light through the blood vessel The drive circuit drives the light-emitting element, based on an inputted drive signal. The detection circuit converts light received by the light-receiving element to an electric signal, and outputs the electric signal as the drive signal.

### Effect of the Invention

According to the sleep state monitoring system of the present invention, by use of data of radial arterial pressure and data of a fingertip vascular pulse wave signal, it is possible to measure blood pressure with higher accuracy and extremely simple calibration as compared with the prior art, and to determine a sleep state with higher accuracy.

### BRIEF DESCRIPTION OF THE DRAWINGS

FIG. 1 is a block diagram showing a configuration of a sleep state monitoring system 10 according to one embodiment of the present invention.

FIG. 2 is a perspective view at the time when a pulse wave and pressure detection application apparatus 20 used for the sleep state monitoring system 10 of FIG. 1 is mounted on a radial arterial portion of a wrist 8, and an optical probe circuit 120 is mounted on a fingertip portion 9.

FIG. 3 is a side view showing a configuration of a reflective optical probe 112 of the optical probe circuit 120 of FIG. 1.

FIG. 4 is a circuit diagram showing a configuration of the optical probe circuit 120 of FIG. 1.

FIG. 5A is a longitudinal sectional view showing a configuration of the pulse wave and pressure detection application apparatus 20 of FIG. 1 provided with a pressure actuator 36 and a MEMS pressure sensor 30.

FIG. 5B is a bottom view of the pulse wave and pressure detection application apparatus 20 of FIG. 5A.

FIG. 6 is a flowchart showing a blood pressure value calibration process executed by a blood pressure value calibration process module 52 of the sleep state monitoring system 10 of FIG. 1.

FIG. 7 is a determination table showing a pattern for sleep state determination executed by a sleep state determination process module 53 of the sleep state monitoring system 10 of FIG. 1.

FIG. 8 is a flowchart showing a sleep state determination process executed by the sleep state determination process module 53 of the sleep state monitoring system 10 of FIG. 1.

FIG. 9 is a block diagram showing a configuration of a sleep state monitoring system 10A according to Modified Embodiment 1 of the present invention.

FIG. 10 is a bottom view of a pulse wave and pressure detection application apparatus 20A according to Modified Embodiment 2 of the present invention.

### MODES FOR CARRYING OUT THE INVENTION

Hereinafter, embodiments according to the present invention will be described with reference to the drawings. It is noted that a similar component in each embodiment below is denoted by the same reference character. Although a pulse wave of a blood vessel of a human will be described below as a measurement target, it may simply be a pulse wave of a blood vessel of an organism, and an animal or the like other than the human can be the target. In addition, although measurement of a pulse, the maximum blood pressure, and the minimum blood pressure will be described below as vascular pulse wave measurement, other than this, measurement using a pulsation waveform of a blood vessel may simply be performed. For example, measurement of an amount corresponding to an amount of blood flow may be performed from an integrated value of a pulse waveform, and measurement for assessing the flexibility of a blood vessel may be performed from a derivative value of the pulsation waveform. Materials, shapes, and the like described below are exemplary, and the contents may be changed as appropriate in accordance with the intended use.

In the present embodiment, there is provided an instrument based on a request for developing a portable sleep monitor capable of grasping a sleep state without measuring brain waves by observing the sleep state not depending on brain waves but by synchronous observation of non-invasive continuous radial arterial pressure and a continuous erasure arterial wave with another respiratory parameter, an electrocardiogram, and the like.

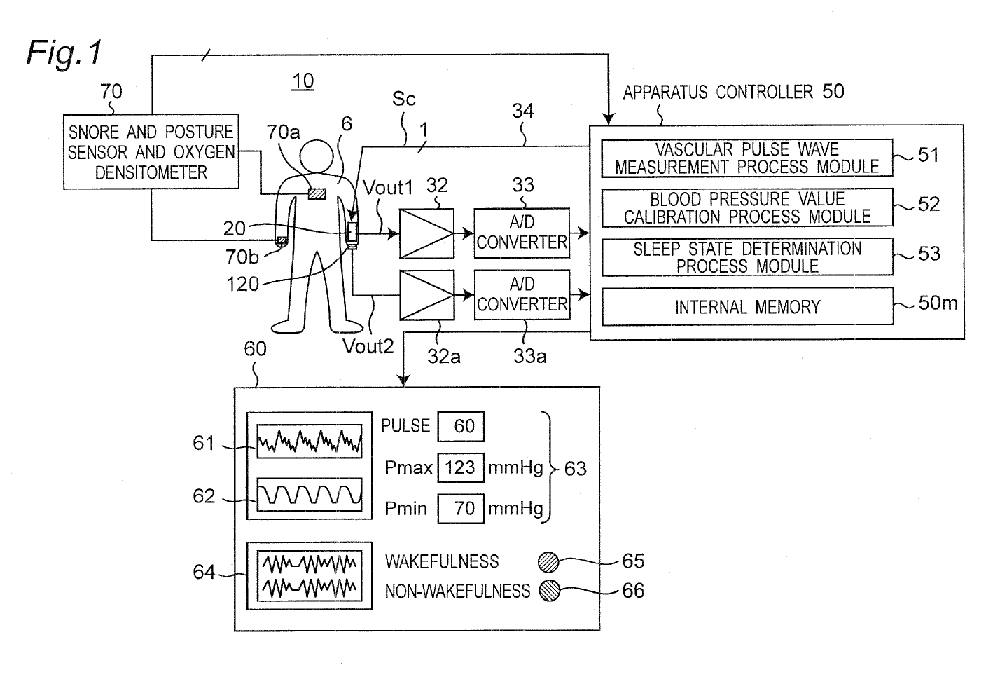

FIG. 1 is a block diagram showing a configuration of a sleep state monitoring system 10 according to one embodiment of the present invention.

FIG. 1 shows a person 6 to be measured who is a target for measurement of blood pressure and the like, although the person is not a component of the sleep state monitoring system 10. It is noted that the skin of the person 6 to be measured is omitted to be shown in the following drawings. The sleep state monitoring system 10 according to the present embodiment is characterized by discriminating a sleep state by use of:

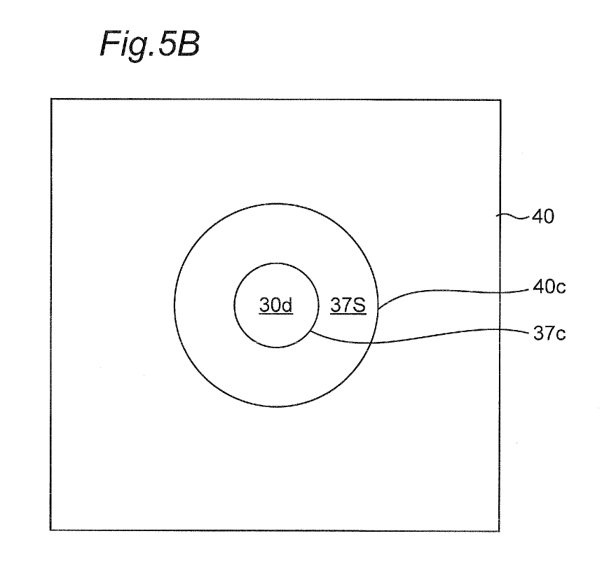

(1) a radial arterial pressure measuring system for acquiring data of radial arterial pressure by use of a MEMS (Micro Electro Mechanical Systems) pressure sensor 30 (FIGS. 5A and 5B) in a pulse wave and pressure detection application apparatus 20 to measure radial arterial pressure as shown in FIG. 1, in place of a conventionally used pressure cuff method of measuring Korotkoff sounds, or a conventionally used invasive method of allowing insertion and invasion of a catheter coupled with a pressure sensor into an artery to directly measure pressure in a blood vessel; and

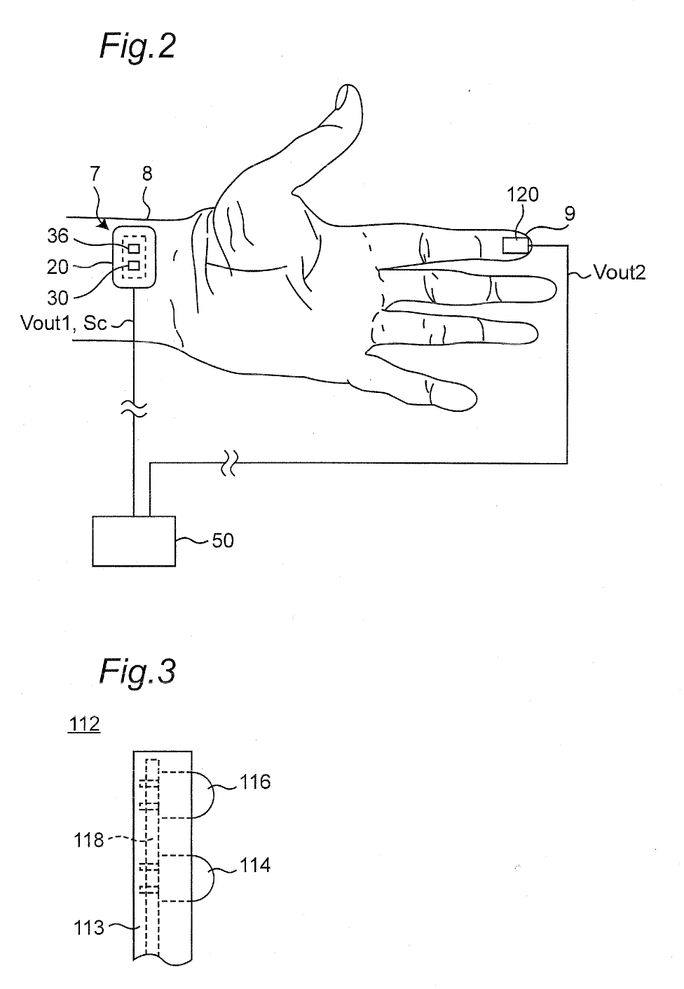

(2) a fingertip blood vessel wave measuring system for measuring a fingertip pulse wave signal by use of an optical probe circuit 120 constituting an optical sensor mounted on a fingertip portion 9 (FIG. 2).

Referring to FIG. 1, the sleep state monitoring system 10 is configured to include the following:

(a) the pulse wave and pressure detection application apparatus 20 that is provided with the MEMS pressure sensor 30 and a pressure actuator 36, and attached to a region suitable for acquiring blood pressure of the person 6 to be measured, such as a region of a radius of the wrist 8, to measure radial arterial pressure;

(b) a voltage amplifier 32 for amplifying an output voltage Vout1 from the MEMS pressure sensor 30 of the pulse wave and pressure detection application apparatus 20;

(c) an A/D converter 33 for A/D converting the output voltage Vout1 from the voltage amplifier 32 to digital data, to output the digital data to an apparatus controller 50;

(d) a control signal line 34 for outputting a control signal Sc from the apparatus controller 50 to the pressure actuator 36 of the pulse wave and pressure detection application apparatus 20;

(e) an optical probe circuit 120 of an optical sensor that is attached to a region suitable for acquiring a pulse wave signal of the person 6 to be measured, such as a region of the fingertip portion 9, to measure a pulse wave signal;

(f) a voltage amplifier 32a for amplifying an output voltage Vout2 from the optical probe circuit 120;

(g) an A/D converter 33a for A/D converting the output voltage Vout2 from the voltage amplifier 32a to digital data, to output the digital data to the apparatus controller 50;

(h) a snore and posture sensor and oxygen densitometer 70 (which is an auxiliary measurement apparatus, and not required to be provided in the sleep state monitoring system) for measuring a snore, a posture, and an oxygen concentration by use of known sensors 70a, 70b, to output the measured values to the apparatus controller 50;

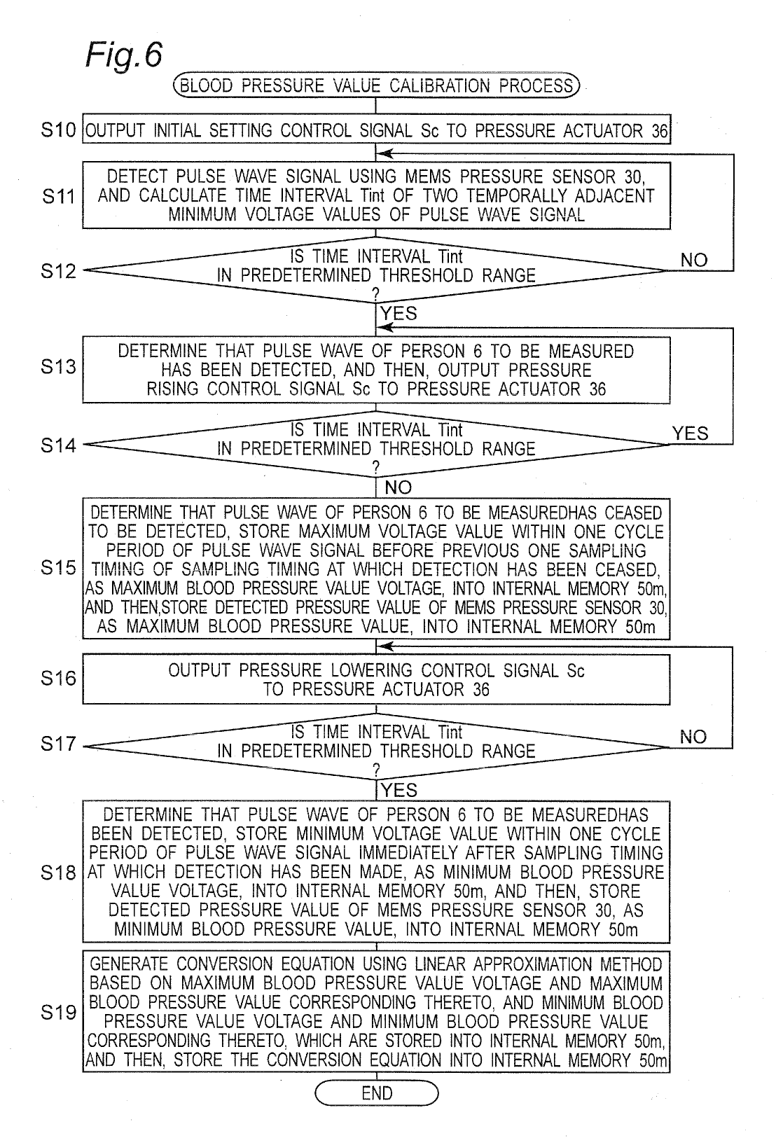

(i) the apparatus controller 50 that is a control apparatus such as a digital calculator which includes an internal memory 50m, is provided with a vascular pulse wave measurement process module 51, a blood pressure value calibration process module 52, and a sleep state determination process module 53, and processes digital data from the A/D converters 33, 33a to generate data of radial arterial pressure and data of a fingertip pulse wave signal and to conduct a blood pressure value calibration process (FIG. 6), a vascular pulse wave measurement process, and a sleep state determination process (FIG. 8); and

(j) a display unit 60 that is, for example, a display (or a printer), and includes a pulsation waveform display 61, an arterial pressure 62, a display 63 of a variety of measured values (a pulse, a maximum blood pressure value Pmax, and a minimum blood pressure value Pmin), a snore waveform and posture sensor waveform display 64, a light emission diode (LED) 65 showing wakefulness, and a light emission diode (LED) 66 showing non-wakefulness, which are displayed based on output data from the apparatus controller 50.

Referring to FIG. 1, an output voltage signal (alternating current (AC)) from the MEMS pressure sensor 30 of the pulse wave and pressure detection application apparatus 20 is outputted to the apparatus controller 50 via the amplifier 32 and the A/D converter 33. In this case, when the blood vessel changes due to pulsation, the AC output voltage Vout1 from the MEMS pressure sensor 30 changes, that is, the output voltage Vout1 changes in response to a change in pulsation. It is noted that, as for measurement of pressure to the blood vessel in the blood pressure value calibration process of FIG. 6, a temporal average value (temporal integrated value) of the output voltage Vout1 from the MEMS pressure sensor 30 is measured to measure applied pressure to the blood vessel. In addition, an output voltage signal (AC) from the optical probe circuit 120 is outputted as a pulse wave signal to the apparatus controller 50 via the amplifier 32a and the A/D converter 33a.

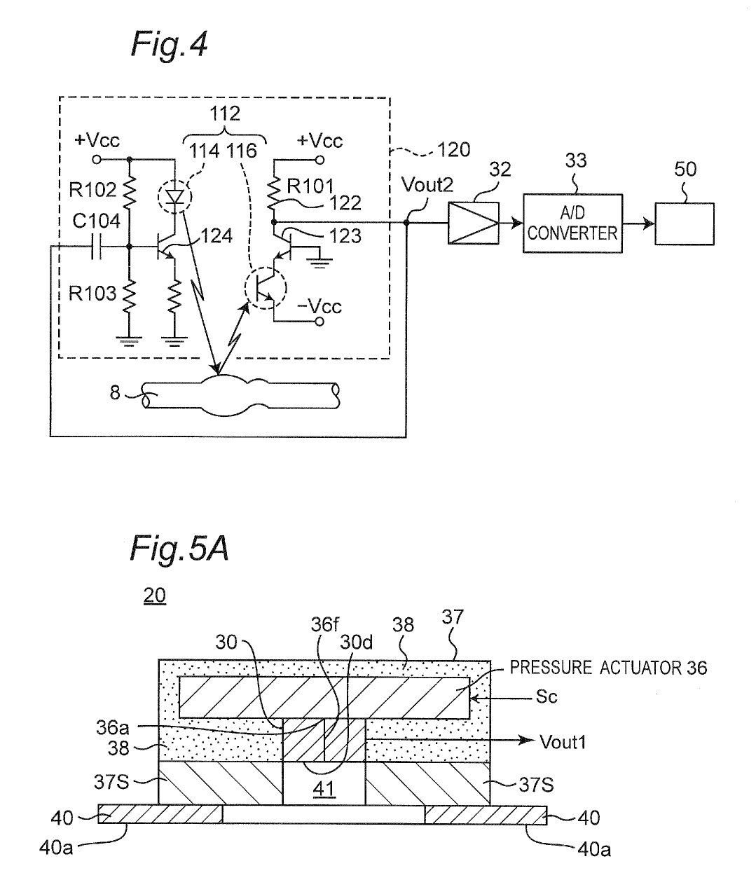

FIG. 2 is a perspective view at the time when the pulse wave and pressure detection application apparatus 20 used for the sleep state monitoring system 10 of FIG. 1 is mounted on a radial arterial portion 7 of the wrist 8, and the optical probe circuit 120 is mounted on the fingertip portion 9. In the pulse wave and pressure detection application apparatus 20 which will be described later in detail with reference to FIGS. 5A and 5B, the MEMS pressure sensor 30 is provided with the pressure actuator 36, generates a voltage signal of the radial arterial pressure, and outputs the voltage signal to the apparatus controller 50. In addition, the optical probe circuit 120 (see FIGS. 3 and 4) detects the fingertip pulse wave signal of the fingertip portion 9 and outputs the fingertip pulse wave signal to the apparatus controller 50.

FIG. 3 is a side view showing a configuration of a reflective optical probe 112 of the optical probe circuit 120 of FIG. 1. Referring to FIG. 3, the optical probe 112 is configured such that a light-emitting element 114 and a light-receiving element 116 are attached to a circuit board 118 and disposed on a predetermined holding unit 113. The holding unit 113 is a member that contains the circuit board 118 and disposes a light radiation unit of light-emitting element 114 and a light detection unit of light-receiving element 116 in a projecting manner. The holding unit 113 is formed by shaping an appropriate plastic material, for example. As the light-emitting element 114, a light emission diode (LED) is usable, and for example, a red LED is used. In addition, as the light-receiving element 116, a photo diode or a photo transistor is used.

While the light-emitting element 114 and the light-receiving element 116 are preferably disposed close to each other, structural improvement such as provision of a shielding wall therebetween is preferably made so as to prevent direct entry of light from the light-emitting element 114 into the light-receiving element 116. Alternatively, a lens may be provided in each of the light-emitting element 114 and the light-receiving element 116 to enhance directivity. In the example of FIG. 3, one light-emitting element 114 and one light-receiving element 116 are provided, but a plurality of light-emitting element 114 and a plurality of light-receiving element 116 may be provided. In addition, a plurality of light-emitting elements 114 may be disposed so as to surround the periphery of a light-receiving element 116. In the optical probe 112, these elements are attached to a region suitable for detecting the pulse wave of the fingertip portion 9 of the person 6 to be measured by use of an appropriate band, tape, or the like, not shown.

FIG. 4 is a circuit diagram showing a configuration of the optical probe circuit 120 of FIG. 1. Referring to FIG. 4, the optical probe circuit 120 is configured of a drive circuit with respect to the light-emitting element 114 and a detection circuit with respect to the light-receiving element 116. The optical probe circuit 120 directly inputs an output signal from the detection circuit to the drive circuit for synchronous feedback, to constitute a self-oscillation circuit.

As the drive circuit with respect to the light-emitting element 114, there is used a configuration where the light-emitting element 114 and a drive transistor 124 are connected in series between a power-supply voltage Vcc and a ground, and a base that is a control terminal of the drive transistor 124 which is made in a predetermined bias condition. In this configuration, when an input signal to the base of the drive transistor 124 becomes high, the drive transistor 124 is turned on, and a drive current flows in the light-emitting element 114. The light-emitting element 114 thus emits light, and the light is emitted toward a blood vessel 8 via the skin. In addition, as the detection circuit for the light-receiving element 116, there is used a configuration where a load resistor 122, a transistor 123, and the light-receiving element 116 are connected in series between a positive power-supply voltage Vcc and a negative power-supply voltage −Vcc. In this configuration, the light-receiving element 116 receives reflected light (transmitted light) from the blood vessel 8, irradiated with light from the light-emitting element 114, via the skin to generate a photocurrent at the light-receiving element 116. A magnitude of the photocurrent is outputted as a signal (output voltage signal) of an output voltage Vout2 corresponding to a magnitude of a current that flows in the load resistor 122. It is noted that the signal of the output voltage Vout2 is a self-oscillation signal, and is thus an AC signal.

The output voltage signal from the optical probe circuit 120 constituting the above self-oscillation circuit is outputted to the apparatus controller 50 via the voltage amplifier 32 and the A/D converter 33. As thus described, when the blood vessel 8 (to be precise, for example, a vascular wall of a blood vessel filled with blood containing oxyhemoglobin) emits light from the light-emitting element 114 and the light-receiving element 116 receives the reflected light from the blood vessel 8, assuming that there is no effect of the light directly incident on the light-receiving element 116 from the light-emitting element 114, as for the output voltage signal from the optical probe circuit 120, the output voltage Vout2 changes in accordance with a propagation distance of light (a distance of light from the light-emitting element 114 to the light-receiving element 116). Accordingly, when the blood vessel 8 changes due to pulsation, the output voltage Vout2 changes, that is, the output voltage Vout2 changes in response to a change in pulsation.

In the prior art, due to the impossibility to obtain a large change in output voltage, a change in frequency has been converted to a change in voltage to detect a change in pulsation. However, in the present embodiment, as shown in FIG. 4, an output signal of the detection circuit in the optical probe circuit 120 is directly synchronously fed back as an input signal of the drive circuit and self-oscillated to generate a self-oscillation signal, which is controlled and set such that the output voltage Vout2 (the width (an amount of change) of the amplitude of the self-oscillation signal of an AC signal) becomes substantially the maximum, making it possible to extremely easily obtain a pulsation waveform.

FIG. 5A is a longitudinal sectional view showing a configuration of the pulse wave and pressure detection application apparatus 20 of FIG. 1 provided with the pressure actuator 36 and the MEMS pressure sensor 30. FIG. 5B is a bottom view of the pulse wave and pressure detection application apparatus 20. Referring to FIGS. 5A and 5B, a housing 37 is formed on a housing substrate 37S having a circular hole 37c. An adhesive sheet 40 of a film sheet is stuck onto the lower surface of the housing substrate 37S, and a lower surface 40a of the adhesive sheet 40, having a circular hole (air hole) 40c and a thickness of 0.5 mm to 1 mm, is made to adhere to the skin of the radial arterial portion 7 of the wrist 8. In an implementation example, a diameter of the circular hole 37c is 1 mm, a diameter of the circular hole 40c is 3 mm, and the adhesive sheet 40 has a size of 4×4 mm. In this case, a diaphragm 30d on the lower surface of the MEMS pressure sensor 30 and the circular holes 37c, 40c are formed so as to be substantially concentric to each other. In addition, by use of the adhesive sheet 40 having a larger area than that of the MEMS pressure sensor 30, the adhesive sheet 40 is reliably stuck onto the skin of the human body.

In the pulse wave and pressure detection application apparatus 20 configured as described above, the circular hole 40c is formed in the adhesive sheet 40 having a larger area than that of the MEMS pressure sensor 30, and pulse pressure from the radial arterial portion 7 of the wrist 8 is transmitted to the diaphragm 30d of the MEMS pressure sensor 30 via a space 41 formed by the circular holes 37c, 40c. Accordingly, even when the radial arterial portion 7 of the wrist 8 is slightly displaced from the centers of the circular holes 37c, 40c, a margin of the position of the MEMS pressure sensor 30 can be made large, to reliably obtain the pulse pressure from the radial arterial portion 7 of the wrist 8. It is noted that pressure transmission medium, such as a gel sheet, may be used in place of the adhesive sheet 40.

FIG. 6 is a flowchart showing a blood pressure value calibration process executed by the blood pressure value calibration process module 52 of the sleep state monitoring system 10 of FIG. 1, and the maximum blood pressure value and the minimum blood pressure value are calibrated by use of similar principles to the cuff pressure method according to the prior art.

Referring to FIG. 6, first of all, an initial setting control signal Sc is outputted to the pressure actuator 36. Subsequently, in step S11, a pulse wave signal is detected using the MEMS pressure sensor 30, and a time interval Tint of two minimum voltage values of the pulse wave signal is calculated, where the two minimum voltage values are temporally adjacent to each other. In step S12, it is determined whether the time interval Tint is in a predetermined threshold range (i.e., it is determined whether a pulse wave signal has been detected), and the process flow goes to step S13 when the determination is YES, whereas the process flow returns to step S11 when it is NO. In this case, the predetermined threshold range of the time interval Tint is a determination range of whether the pulse wave signal has been detected, and the threshold range is, for example, 0.2 seconds≦Tint≦2 seconds as an empirical value. When the time interval Tint is in the threshold range, it is determined that a pulse wave has been detected. In step S13, it is determined that the pulse wave of the person 6 to be measured has been detected, and a pressure rising control signal Sc is outputted to the pressure actuator 36 in order to increment the pressure by a predetermined differential pressure. Then, in step S14, it is determined whether the time interval Tint is in the predetermined threshold range (i.e., it is determined whether the pulse wave signal has been detected), and the process flow goes to step S15 when the determination is NO, whereas the process flow returns to step S13 when it is YES.

In step S15, it is determined that the pulse wave of the person 6 to be measured has ceased to be detected, and the maximum voltage value within one cycle period of the pulse wave signal before the previous sampling timing of the sampling timing at which the detection has been ceased is stored as the maximum blood pressure value voltage into the internal memory 50m. Along with this, a detected pressure value of the MEMS pressure sensor 30 is stored as the maximum blood pressure value into the internal memory 50m. Then, in step S16, a pressure lowering control signal Sc is outputted to the pressure actuator 36 in order to decrement the pressure by a predetermined differential pressure. Subsequently, in step S17, it is determined whether the time interval Tint is in the predetermined threshold range (i.e., it is determined whether the pulse wave signal has been detected), and the process flow goes to step S18 when the determination is YES, whereas the process flow goes back to step S16 when it is NO. In step S18, it is determined that the pulse wave of the person 6 to be measured has been detected, and the minimum voltage value within one cycle period of a pulse wave signal immediately after the sampling timing at which the pulse wave has been detected is stored as the minimum blood pressure value voltage into the internal memory 50m. Along with this, a detected pressure value of the MEMS pressure sensor 30 is stored as the minimum blood pressure value into the internal memory 50m. In addition, in step S19, based on the maximum blood pressure value voltage and the maximum blood pressure value corresponding thereto and the minimum blood pressure value voltage and the minimum blood pressure value corresponding thereto which are stored into the internal memory 50m, a conversion equation (or a blood pressure conversion table) showing conversion from a voltage value to a blood pressure value is generated using a linear approximation method, as described with reference to FIG. 8C, and the generated conversion equation is stored into the internal memory 50m. The present process is thus completed.

The blood pressure value calibration process of FIG. 6 has been executed using, for example, the pulse wave and pressure detection application apparatus 20 of FIG. 2, but the present invention is not limited thereto, and it may be executed only using the MEMS pressure sensor 30. In this case, in step S13, it is determined that the pulse wave of the person 6 to be measured has been detected, and the pressure actuator 36 of a pressure application mechanism is not used, but a message is displayed on an LCD display unit (not shown), the message instructing the human, such as a person to be tested, to press the top of the MEMS pressure sensor 30 by the fingertip portion 9. At this time, the human presses the MEMS pressure sensor 30 by the fingertip portion 9. In addition, in step S16, it is determined that the pulse wave of the person 6 to be measured has ceased to be detected, and the pressure actuator 36 of the pressure application mechanism is not used, but a message is displayed on the LCD display unit (not shown), the message instructing the human, such as the person to be tested, to loose and decrease the above stress applied by the fingertip portion 9. At this time, the human loosens the pressing force of the fingertip portion 9. In this manner, the pressure actuator 36 of the pressure application mechanism can be substituted by the fingertip portion 9 of the human such as the person to be tested. Further, the calibration may be made by pressing force through use of a cuff of a cuff-sphygmomanometer in place of the pressing force by the pressure actuator 36 or the fingertip portion 9 of the human. In addition, the maximum and minimum blood pressure values, separately measured by the cuff-sphygmomanometer, may be manually inputted as calibration values. It is noted that not needing the pressure actuator 36 will be described later in detail.

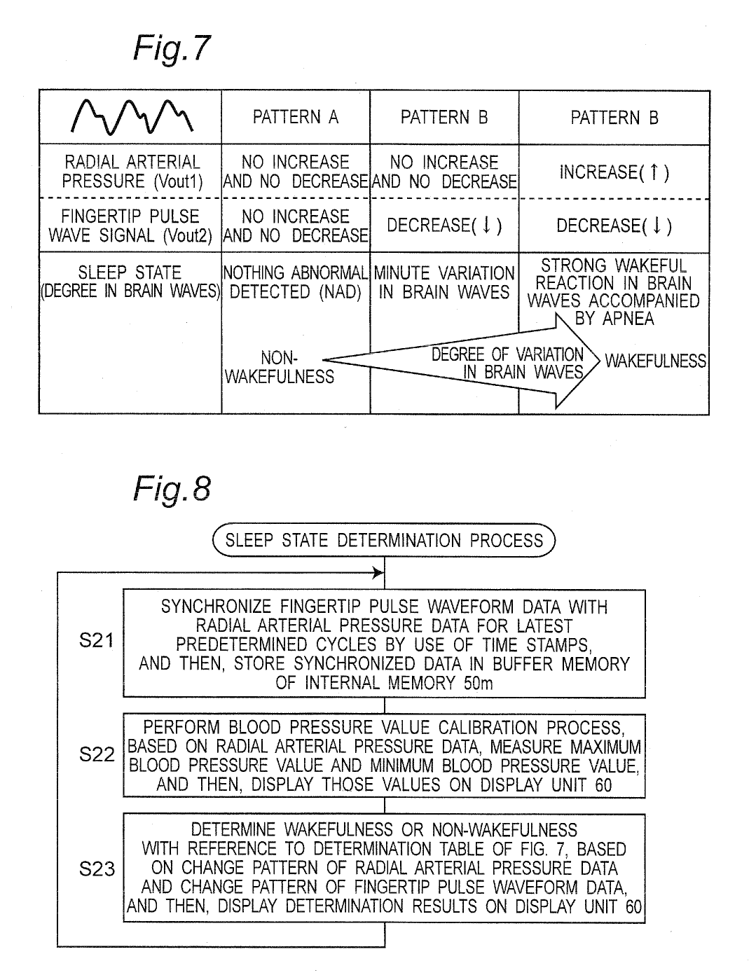

FIG. 7 is a determination table showing a pattern for sleep state determination executed by the sleep state determination process module 53 of the sleep state monitoring system 10 of FIG. 1. In the determination table of FIG. 7, a message “NO INCREASE AND NO DECREASE” means that each of the radial arterial pressure (Vout1) and the fingertip pulse wave signal (Vout2) does not increase or decrease by an amount of increase or decrease equal to or larger than a predetermined threshold, and does not increase or decrease by an amount of increase or decrease equal to or smaller than the predetermined threshold, that is, increases or decreases by an amount of increase or decrease within a predetermined threshold range. The term “increase” refers to an increase by an amount of increase equal or larger than the predetermined threshold, and the term “decrease” refers to a decrease by an amount of decrease equal to or smaller than the predetermined threshold.

In the determination table of FIG. 7, the sleep state is determined as follows:

(1) Pattern A: When the radial arterial pressure (Vout1) is “NO INCREASE OR DECREASE” and the fingertip pulse wave signal (Vout2) is also “NO INCREASE AND NO DECREASE”, the sleep state is determined to be “NOTHING ABNORMAL DETECTED (NAD)”, and determined to be “NON-WAKEFULNESS”. At this time, the apparatus controller 50 lights the light emission diode 66.

(2) Pattern B: When the radial arterial pressure (Vout1) is “NO INCREASE OR DECREASE” and the fingertip pulse wave signal (Vout2) is “DECREASE”, the sleep state is determined to be “STATE WHERE MINUTE VARIATION OF BRAIN WAVES IS RECOGNIZED”, and determined to be a state which is neither “NON-WAKEFULNESS” nor “WAKEFULNESS”.

(2) Pattern C: When the radial arterial pressure (Vout1) is “INCREASE” and the fingertip pulse wave signal (Vout2) is “DECREASE”, the sleep state is determined to be “STATE WHERE STRONG WAKEFUL REACTION OF BRAIN WAVES, ACCOMPANIED BY APNEA IS RECOGNIZED”, and determined to be “WAKEFULNESS”. At this time, the apparatus controller 50 lights the light emission diode 65.

FIG. 8 is a flowchart showing the sleep state determination process executed by the sleep state determination process module 53 of the sleep state monitoring system 10 of FIG. 1.

Referring to FIG. 8, first of all, in step S21, fingertip pulse waveform data and radial arterial pressure data for the latest predetermined cycle (four to ten beats) are synchronized with each other by use of time stamps, which are then stored in a buffer memory of the internal memory 50m. Subsequently, in step S22, the blood pressure value calibration process is performed based on the radial arterial pressure data, to measure the maximum blood pressure value and the minimum blood pressure value and display those values on the display unit 60. Further, in step S23, based on a change pattern of the radial arterial pressure data and a change pattern of the fingertip pulse waveform data, the wakefulness or the non-wakefulness is determined with reference to the determination table of FIG. 7, and displayed on the display unit 60. The above process is repeated over a predetermined cycle.

As described above, according to the present embodiment, the increase and decrease patterns of the “radial arterial wave” and the “fingertip pulse wave” based on synchronous observation of both waveforms are compared in a manner similar to that of the determination table of FIG. 7, and can thus be classified into a plurality of variation pattern types. This variation pattern is associated with the degree of variation in brain waves (based on clinical data) to determine the sleep state. In addition, respiratory state observation data is also combined to determine the sleep state with higher accuracy.

Although a basic type of combination of the variation patterns of the “radial arterial wave” and the “fingertip pulse wave” is shown in this case, it is possible to determine a more elaborate sleep state by further combining time-base variations in the respective pulse waves with the combination of increase and decrease tendencies of both the patterns. In addition, as one example, the current simple sleep monitoring (without brain wave observation) can be performed. Further, not only a sleep stage (lightness or deepness of sleep) and a PAT (Peripheral Arterial Tone) respiratory event can be assessed, but also parameters such as CAP (Cyclic Alternating Pattern), a wakefulness index, and a respiratory effort can also be assessed. It is noted that the sleep state determination process module as a function of a monitoring process unit may perform a software process on another computer.

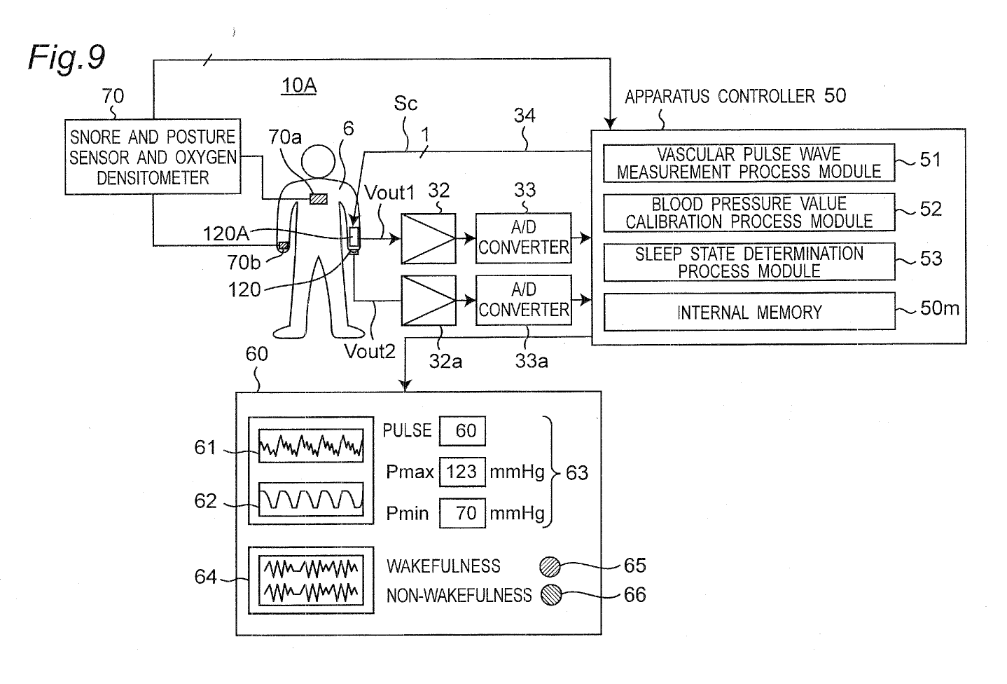

As described in detail above, by use of data of radial arterial pressure and data of a fingertip vascular pulse wave signal, the sleep state monitoring system according to the embodiment of the present invention can measure blood pressure with higher accuracy and extremely simple calibration as compared with the prior art, to determine a sleep state with higher accuracy. As is known, the sleep state changes due to a change in dominancy between the sympathetic nerve and the parasympathetic nerve, and in this case, the sleep state becomes the wakefulness when the sympathetic nerve is dominant, whereas it becomes the non-wakefulness (sleep state) when the parasympathetic nerve is dominant. In the present embodiment, the sleep state is determined using the MEMS pressure sensor 30 for measuring arterial pressure (in Modified Embodiment 1 of FIG. 9 described later, an optical sensor including an optical probe 120A is used), and an optical sensor including the optical probe 120 for measuring a pulse wave that propagates through the peripheral blood vessel. The correlation between these two blood pressure variations and the change in dominancy between the sympathetic nerve and the parasympathetic nerve (which is knowledge uniquely acquired by the present inventors) is hardly different among individuals even when persons to be measured are different, thus exerting a specific effect of being able to determine the sleep state with higher accuracy.

In the above embodiment, the radial arterial pressure has been measured by the MEMS pressure sensor 30, but the present invention is not limited thereto. The radial arterial wave may be measured using the optical probe circuit 120A (see FIG. 9 according to Modified Embodiment 1) which is similar to the optical probe circuit 120 including the optical sensor. In this case, the massage “RADIAL ARTERIAL PRESSURE (Vout1)” in the determination table of FIG. 7 becomes a message “RADIAL ARTERIAL WAVE SIGNAL”. Also using this, the sleep state can be determined based on determination of the patterns A, B, or C of FIG. 7.

Although the pulse wave and pressure detection application apparatus 20 is provided in the above embodiment, when only the sleep state is to be determined without performing the blood pressure measurement, the MEMS pressure sensor 30 for measuring only arterial pressure may only be provided and the pressure actuator 36 may not be provided.

Although the sleep state is determined using data of radial arterial pressure and data of a fingertip vascular pulse wave signal in the above embodiment, the present invention is not limited thereto. The sleep state may be determined by measuring data of aortic pressure of an upper arm or the like as the former data, and measuring data of a pulse wave signal of a peripheral blood vessel, such as a microvasculature of an ear or the like, as the latter data.

In addition, in the sleep state monitoring system 10 of FIG. 1, the display is performed such that the light emission diode 65 is lighted when the sleep state is the wakefulness and the light emission diode 66 is lighted when the sleep state is the non-wakefulness, but the present invention is not limited thereto. The sleep state may be notified by a sound, a voice, or a vibration, or reported by outputting data that indicates the sleep state to an external circuit.

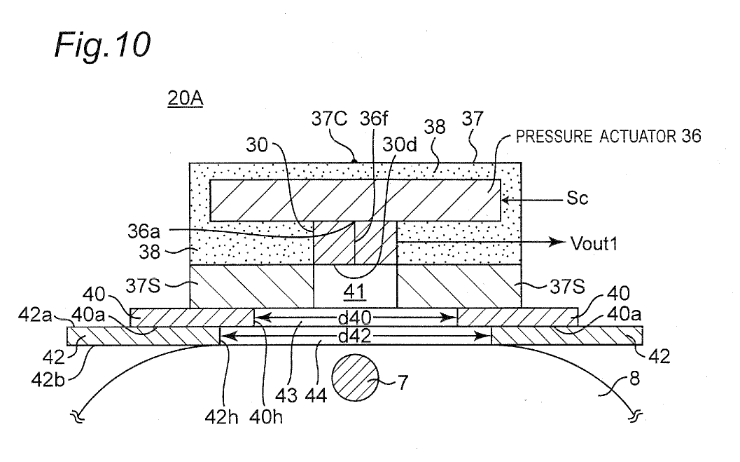

FIG. 10 is a bottom view of a pulse wave and pressure detection application apparatus 20A according to Modified Embodiment 2 of the present invention.

Referring to FIG. 10, the pulse wave and pressure detection application apparatus 20A according to Modified Embodiment 2 is different from the pulse wave and pressure detection application apparatus 20 of FIG. 5A in the following respects:

(1) At a central portion of an adhesive sheet 40 of a film sheet, a space 43 is formed by a pressure detecting hollow hole 40h.

(2) In order to position a pressure sensor 30 (in the pulse wave and pressure detection application apparatus 20A) having the adhesive sheet 40, there is further provided an adhesive sheet 42 of a film sheet that previously adheres to a radial arterial portion 7 of a wrist 8. At a central portion of the adhesive sheet 42, a space 44 having a size in a parallel direction to a pressure detection surface is formed by a pressure detecting hollow hole 42h. In this case, in order to facilitate sticking of the adhesive sheet 42 onto the radial arterial portion 7, a diameter d42 of the pressure detecting hollow hole 42h is larger than a diameter d40 of the pressure detecting hollow hole 40h of the adhesive sheet 40. In this configuration example, d42=5 mm, and d40=3 mm. In addition, spaces 41, 43, and 44 constitute the sealed spaces described in the embodiment.

(3) A mark 37C indicating the center is preferably drawn at a central portion of an upper surface of a housing 37 of the pressure sensor 30.

Subsequently, a procedure for a method of positioning the pressure sensor 30 by use of the adhesive sheet 40 will be described.

For detecting blood vessel pulsation at a higher S/N ratio in a pulse wave measuring system using the MEMS pressure sensor 30, the pressure sensor 30 needs to be precisely disposed on the radial arterial portion 7 of the wrist 8 of the person to be observed. The following procedure is used in order to effectively conduct this operation.

(Step A) First of all, the adhesive sheet 42 is stuck onto a position of the radial arterial portion 7 (a place, first confirmed as a position where a pulse can be taken and then marked, is preferred). In this case, the adhesive sheet 42 is stuck as positioned such that an adhesive lower surface 42b of the adhesive sheet 42 adheres to the skin surface of the radial arterial portion 7, and a central portion of the pressure detecting hollow hole 42h of the adhesive sheet 42 is located in the radial arterial portion 7.

(Step B) Subsequently, an upper surface 42a of the adhesive sheet 42 is stuck onto the adhesive sheet 40. In this case, the pressure sensor 30 having the adhesive sheet 40 is positioned such that the mark 37C is located at a central portion of the pressure detecting hollow hole 42h, that is, the central portion of the pressure detecting hollow hole 42h of the adhesive sheet 42 is located at a central portion of the pressure detecting hollow hole 40h.

As described above, by the two stages of sticking step, it is possible to reliably form the sealed spaces 41, 43, and 44 for detecting pulsation by use of the MEMS pressure sensor 30. It is noted that in Modified Embodiment 1, in a manner similar to the embodiment, when the pressure sensor 30 is placed in a portion to be measured, the spaces 41, 43, and 44 are sealed to become sealed spaces, and pressure of the portion to be measured is transmitted to a diaphragm 30d of the MEMS pressure sensor 30 via the spaces 41, 43, 44, and the MEMS pressure sensor 30 detects the pressure. Therefore, even when the position of the MEMS pressure sensor 30 is displaced from a measurement position, the pressure of the portion to be measured can be precisely measured. In addition, since the pressure does not need to be applied to the portion to be measured, it is possible to measure, for example, a non-invasive blood pressure pulse wave over a long period of time.

### INDUSTRIAL APPLICABILITY

As mentioned above in details, according to the sleep state monitoring system of the present invention, by use of data of radial arterial pressure and data of a fingertip vascular pulse wave signal, it is possible to measure blood pressure with higher accuracy and extremely simple calibration as compared with the prior art, and to determine a sleep state with higher accuracy.

According to the sleep state monitoring system of the present invention, a sleep state can be observed even by a simple sleep test by use of a portable sleep monitor with a non-invasive blood pressure monitoring function which enables a test in accordance with the PSG capable of observing a respiratory state during sleep. Hence the sleep state monitoring system can be expected not only to diagnose SAS which is predicted to increase in the future, but also to exert a ripple effect in the following fields:

(1) the preventive health field;

(2) the field for children to elderly people (home care); and

(3) the quality of sleep and a dynamic state of a circulatory organ can be assessed in a place other than a laboratory.

In addition, the non-invasive blood pressure continuous monitoring function is a useful function in a blood pressure measurement field, and can be expected to be applied to grasping of the connection between high blood pressure early in the morning and SAS, or applied to continuous blood pressure monitoring in CPAP (Continuous Positive Airway Pressure) treatment for an SAS patient, and the like. In particular, assessing a dynamic state of a circulatory organ by use of continuous blood pressure measurement and monitoring can contribute to the development of a new medicine or the development of a new diagnosis technique.

### DESCRIPTION OF REFERENCE CHARACTERS

6: PERSON TO BE MEASURED

7: RADIAL ARTERIAL PORTION

8: WRIST

9: FINGERTIP PORTION

10, 10A: SLEEP STATE MONITORING SYSTEM

20, 20A: PULSE WAVE AND PRESSURE DETECTION APPLICATION APPARATUS

30: MEMS PRESSURE SENSOR

30d: DIAPHRAGM

32, 32a: VOLTAGE AMPLIFIER

33, 33a: A/D CONVERTER

34: CONTROL SIGNAL LINE

36: PRESSURE ACTUATOR

37: HOUSING

37A: GEL SHEET

37S: HOUSING SUBSTRATE

38: FILLER

38A: GEL

39: CALIBRATION PRESSURE SENSOR

40, 42: ADHESIVE SHEET

41, 43, 44: SPACE

50: APPARATUS CONTROLLER

50m: INTERNAL MEMORY

51: VASCULAR PULSE WAVE MEASUREMENT PROCESS MODULE

52: BLOOD PRESSURE VALUE CALIBRATION PROCESS MODULE

53: SLEEP STATE DETERMINATION PROCESS MODULE

60: DISPLAY UNIT

112: OPTICAL PROBE

113: HOLDING UNIT

114: LIGHT-EMITTING ELEMENT

116: LIGHT-RECEIVING ELEMENT

118: CIRCUIT BOARD

120, 120A: OPTICAL PROBE CIRCUIT

122: LOAD RESISTOR

124: DRIVE TRANSISTOR

1. A sleep state monitoring system comprising:
a first sensor that is one of a pressure sensor and an optical sensor, the first sensor of the pressure sensor being provided via a skin on a blood vessel of an aortic portion of a human body and detecting a change in pressure of a pulse wave propagating through the blood vessel to measure a voltage value as a first voltage value of arterial pressure, the first sensor of the optical sensor being provided via the skin on the blood vessel of the aortic portion of the human body and detecting a pulse wave propagating through the blood vessel to measure a voltage value as a first voltage value of blood vessel pressure;
a second sensor that is an optical sensor, the second sensor being provided via a skin on a peripheral blood vessel of the human body and detecting a change in pressure of a pulse wave propagating through the blood vessel by use of an optical signal to measure a voltage value as a second voltage value of a vascular pulse wave signal; and
a controller configured to determine a sleep state of the human body, based on the first voltage value and the second voltage value,
wherein the controller
(A) determines that the human body is in non-wakefulness when the first voltage value increase and decrease within a first increase and decrease amount and the second voltage value increase and decrease within a second increase and decrease amount for a predetermined time interval, and
(B) determines that the human body is in wakefulness when the first voltage value increases by an amount of increase equal or larger than a predetermined first threshold and the second voltage value decreases by an amount of decrease equal to or smaller than a predetermined second threshold for the time interval.

2. The sleep state monitoring system as claimed in claim 1,
wherein the controller
(C) determines that the human body is in a state of minute variation of brain waves when the first voltage value increases and decreases within the first increase and decrease amount and the second voltage value decreases by an amount of decrease equal to or smaller than the predetermined second threshold range for the time interval.

3. The sleep state monitoring system as claimed in claim 1,
wherein the blood vessel of the aortic portion of the human body is a blood vessel of a radial portion of the human body, and
wherein the peripheral blood vessel of the human body is a blood vessel of a fingertip portion of the human body.

4. The sleep state monitoring system as claimed in claim 1, further comprising a notification device that notifies the determination result.

5. The sleep state monitoring system as claimed in claim 1,
wherein the pressure sensor is a MEMS (Micro Electro Mechanical Systems) pressure sensor that detects, as a change in resistance value, a change in pressure of a pulse wave propagating through the blood vessel.

6. (canceled)

7. The sleep state monitoring system as claimed in claim 1,
wherein the optical sensor is an optical sensor configured using an optical probe circuit, and
wherein the optical probe circuit includes:
an optical probe including a light-emitting element that emits light to a blood vessel via a skin, and a light-receiving element that receives, via a skin, reflected light from the blood vessel and transmitted light through the blood vessel;
a drive circuit that drives the light-emitting element, based on an inputted drive signal; and
a detection circuit that converts light received by the light-receiving element to an electric signal, and outputs the electric signal as the drive signal.
